(19) United States
(12) Patent Application Publication
(10) Pub. No.: US 2006/0003974 A1
(43) Pub. Date: Jan. 5, 2006

(54) Bile acid derived steroidal dimers with amphiphilic topology having antiproliferative activity

(75) Inventors: Deepak Bhalchandra Salunke, Pune (IN); Braja Gopal Hazra, Pune (IN); Vandana Sudhir Pore, Pune (IN); Manoj Kumar Bhat, Pune (IN)

Correspondence Address: LADAS & PARRY, 26 WEST 61ST STREET, NEW YORK, NY, 10023, US

(73) Assignees: COUNCIL OF SCIENTIFIC AND INDUSTRIAL RESEARCH; NATIONAL CENTER FOR CELL SCIENCES

(21) Appl. No.: 11/089,451

(22) Filed: Mar. 24, 2005

Related U.S. Application Data
(63) Continuation of application No. 10/881,728, filed on Jun. 30, 2004.

(51) Int. Cl. A61K 31/56; C07J 1/00

(52) U.S. Cl. 514/169; 552/509

(57) ABSTRACT

Advances in the treatment of cancer required continued development of novel and improved therapeutic agents. A highly efficient modified process for the synthesis of bile acid derived steroidal dimers N¹,N²-Ethylenediamine bis [cholic acid amide] and N¹,N²-Ethylenediamine bis [deoxycholic acid amide] having novel amphiphile topology and remarkable antiproliferative activity is described.

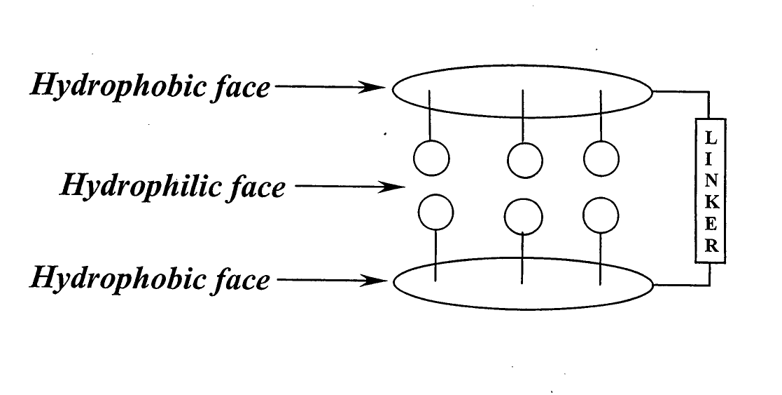

### FIELD OF THE INVENTION

The present invention relates to an improved process for the preparation of compounds N¹,N²-Ethylenediamine bis [cholic acid amide] (2) and N¹,N²-Ethylenediamine bis [deoxycholic acid amide] (3) respectively 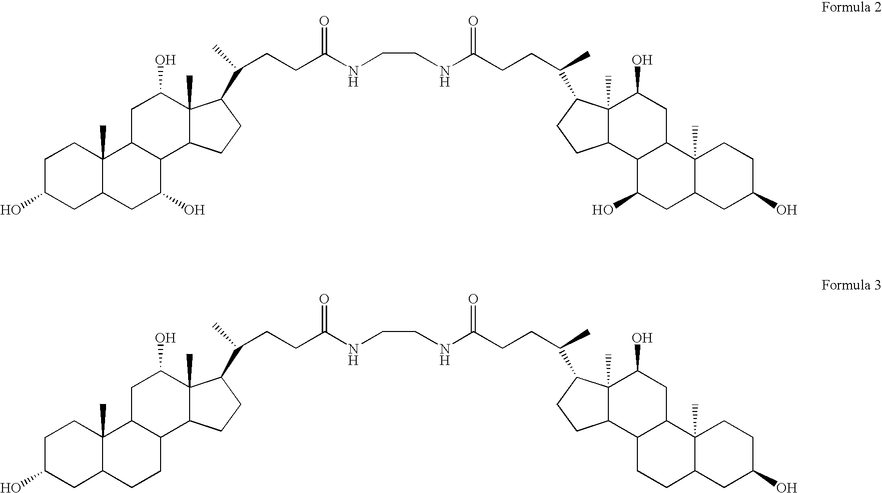
having amphiphilic topology as shown in the FIGURE below with antiproliferative activity.

### BACKGROUND OF THE INVENTION

Despite major advances in prevention and therapy, cancer continues to be one of the major health problems in the world. Advances in the treatment of cancer require the continued development of novel and improved therapeutic agents.

Compounds having structural formula (2) and (3) have been synthesized from N-succinimidyl ester of cholic acid and deoxycholic acid having structural formula (4) and (5) respectively by an improved process.

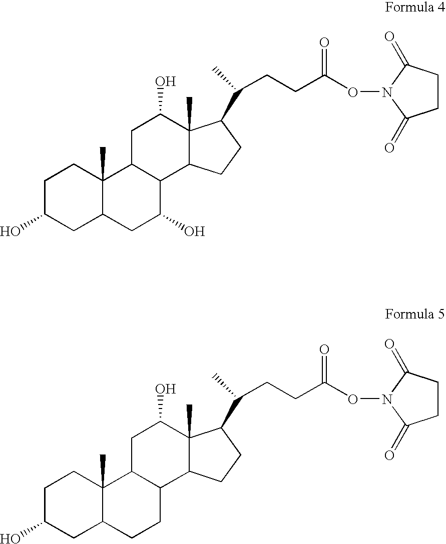

The title compounds having structural formulae (2) and (3) are found to possess amphiphilic topology and show very good antiproliferative activity and the results have been incorporated in this specification. Compound having structural formula (4) and (5) can be prepared from cholic acid or deoxycholic acid and N-hydroxy succinimide in the presence of dicyclohexylcarbodiimide. [Reference: Okahata, Y.; Ando, R.; Kunitake, T. Bull. Soc. Chem. Jpn, 1979, 52, 3647-3653]

### PRIOR ART

Steroidal dimers have tremendous applications in different areas. [Reference: Yuexian Li; Dias, R. Chem. Rev. 1997, 97, 283-304 and Davis, A. P. Chem. Soc. Rev. 1993, 243-253]. There are two reports in which synthesis of compound having formula (1) and (2) have been carried out as an intermediate for the synthesis of cholaphanes. [Reference: Pandey, P. S.; Rai, R.; Singh, R. B. Tetrahedron Letters 1997, 38, 5045-5046; Pandey, P. S.; Rai, R.; Singh, R. B. J. Chem. Soc., Perkin Trans. 1, 2002, 918-923]. In this process compounds (1) and (2) have been synthesized in three steps. Eur. Pat. Application EP 489423 describes a process for the preparation of bile acid derivatives and their use in medicine. In this patent application steroidal dimers have been synthesized as inhibitors of bile acid resorption than the compounds described in the present invention. Furthermore in the above mentioned references amphiphilic topology of compounds (2) and (3) and their biological activity has not been mentioned. Compounds having structural formulae (2) and (3) show amphiphilic topology (FIGURE). The compounds described in this invention show remarkable antiproliferative activity.

### BRIEF DESCRIPTION OF THE FIGURE

The FIGURE shows the amphiphilitic topology of compounds (2) and (3).

### OBJECTS OF THE INVENTION

It is therefore an object of this invention to provide steroidal dimers having structural formulae (2) and (3) showing amphiphilic topology (the FIGURE) and a process for preparation thereof.

Another object is to provide the antiproliferative activity of the said steroidal dimers.

### SUMMARY OF THE INVENTION

The present invention provides steroidal dimers having structural formula (1) 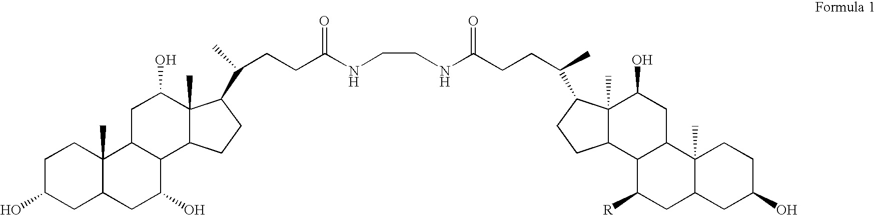
where R is OH or H and possessing amphiphilic topology as shown in the FIGURE below and showing antiproliferative activity

In one embodiment of the invention, in the compound of formula 1 R is OH and the compound has the structural formula (2)

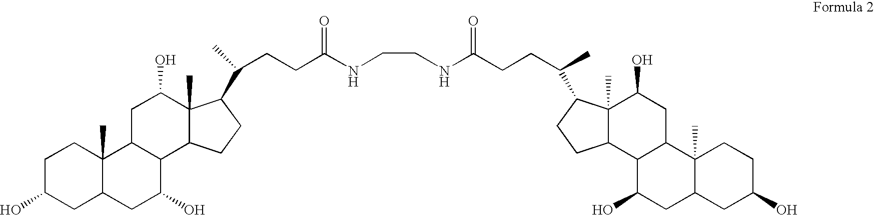

In one embodiment of the invention, in the compound of formula 1 R is OH and the compound has the structural formula (3)

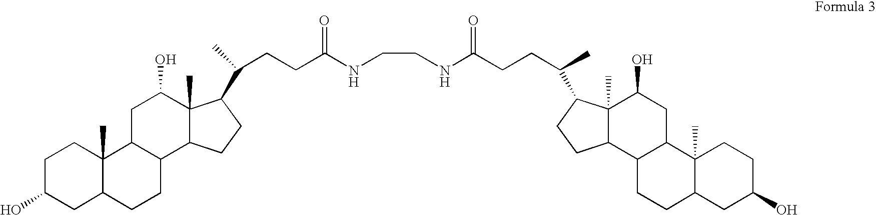

The present invention also provides a process for the preparation of steroidal dimers having structural formula (1) where R is OH or H 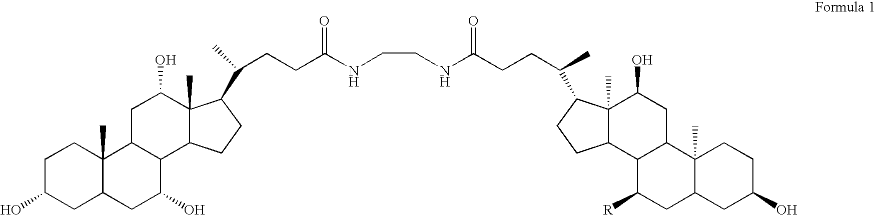
which comprises preparing a solution of N-succinimidyl ester of bile acids in an organic solvent at a temperature ranging between 10 to 50° C., adding to this solution ethylenediamine at a temperature ranging between 10 to 50° C., stirring the reaction mixture for a period of at least 1 h at a temperature ranging from 20 to 70° C., quenching the reaction mixture with ice, filtering the solid to obtain crude dimers having structural formulae (1), further purifying the crude dimers (1) to obtain the pure steroidal dimers (1).

In an embodiment of the invention the bile acid used may be cholic acid, deoxycholic acid.

In one embodiment of the invention, in the compound of formula 1 R is OH and the compound has the structural formula (2)

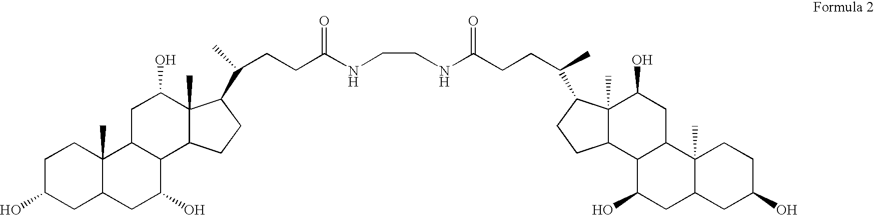

In one embodiment of the invention, in the compound of formula 1 R is H and the compound has the structural formula (3)

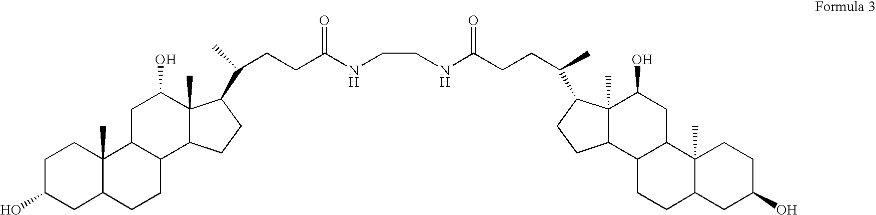

In another embodiment the organic solvents used for the preparation of N-succinimidyl ester solutions may be chlorinated solvents such as chloroform and dichloromethane or polar aprotic solvents such as dimethy formamide or dimethyl sulfoxide.

In another embodiment the crude products (1) are purified by column chromatography using silica gel, basic alumina, neutral alumina as adsorbants and ethylacetate-hexane, ethylacetate, ethylacetate-chloroform, chloroform-methanol as solvent systems.

### DETAILED DESCRIPTION OF THE INVENTION

The present invention provides a process for the preparation of steroidal dimers having structural formula (1) where R is OH or H 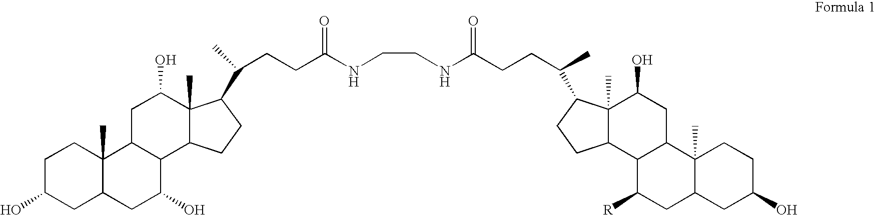
possessing amphiphilic topology as shown in FIGURE (2) and showing antiproliferative activity.

The dimers can be represented by the following structural formulae (2) and (3) respectively

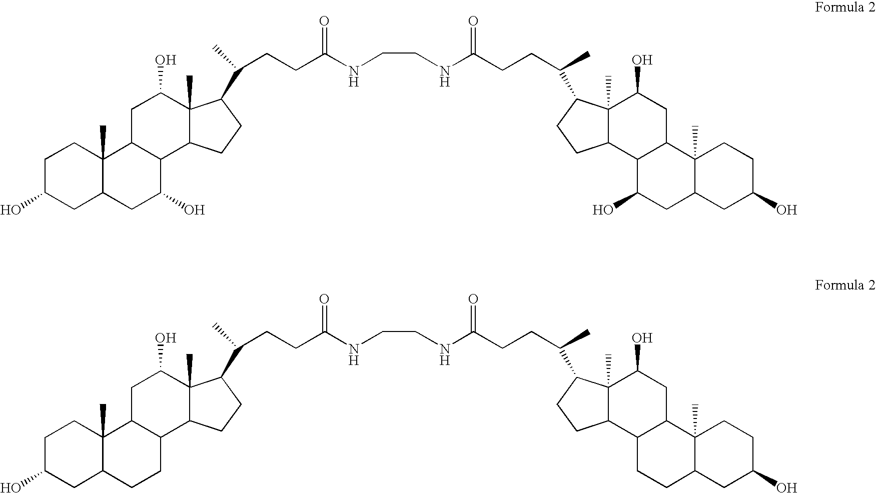

The process comprises preparing a solution of N-succinimidyl ester of bile acids in an organic solvent at a temperature ranging between 10 to 50° C. Ethylenediamine is added to this solution at a temperature ranging between 10 to 50° C. and the mixture stirred for a period of at least 1 h at a temperature ranging from 20 to 70° C. and then quenched with ice. The solid is filtered to obtain crude dimers having structural formulae (2) and (3) which are then purified to obtain the pure steroidal dimers (2) and (3).

The bile acid used may be cholic acid or deoxycholic acid. The organic solvents used for the preparation of N-succinimidyl ester solutions may be chlorinated solvents such as chloroform and dichloromethane or polar aprotic solvents such as dimethy formamide or dimethyl sulfoxide.

In the feature of present invention the crude products (2) and (3) may be purified by column chromatography using silica gel, basic alumina, neutral alumina as adsorbants and ethylacetate-hexane, ethylacetate, ethylacetate-chloroform, chloroform-methanol as solvent systems.

The present invention also provides steroidal dimers having structural formula (1) where R is OH or H. 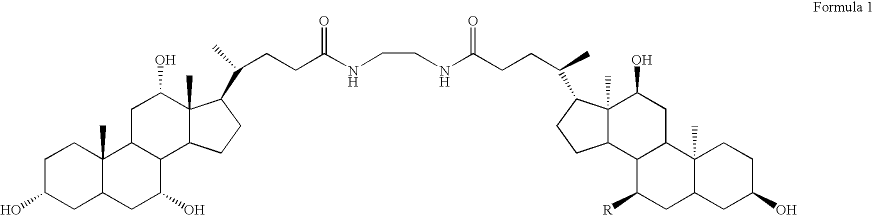
possessing amphiphilic topology as shown in the FIGURE and showing antiproliferative activity

Representative compounds are dimers of formulae (2) and (3) depicted below respectively.

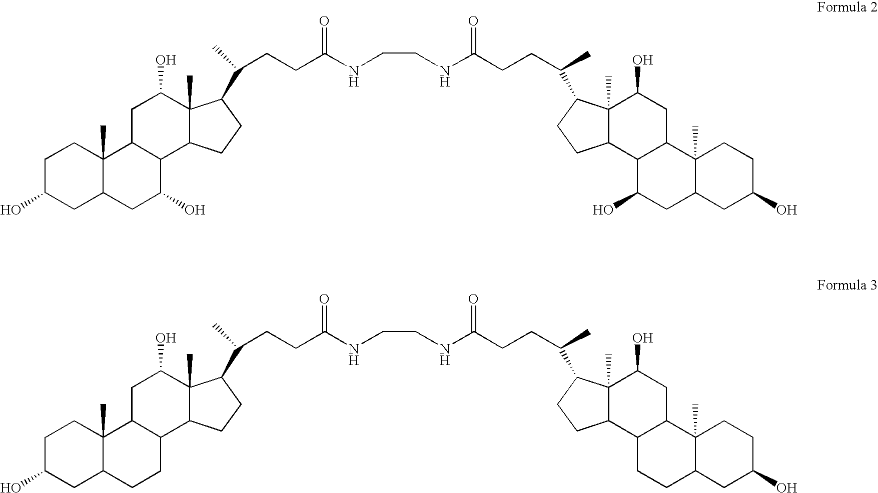

The compounds (2) and (3) of the present invention represents amphiphilic topology that has not previously described, having partially rigid structure with three discrete faces, one polar face sandwiched within two non polar faces as shown in FIGURE shown above. which were grown in vitro. However compound (3) elicited a significant reduction in proliferation of both the cell lines (IC₅₀=2-4 μM) and at 10 μM concentration totally inhibited their growth.

The following examples illustrate several preferred embodiments to describe the invention however it should be construed to limit the scope of the present invention.

### EXAMPLE 1

N-succinimidyl ester (4) (1.01 g, 2 mmol) of cholic acid was dissolved in 2 ml of dimethylformamide. To it ethylenediamine (0.073 ml, 1.1 mmol) was added at 20° C. and reaction mixture was stirred for the period of 5 h at 10° C. Reaction was quenched by the addition of crushed ice. Solid crude product was filtered, washed with cold water and dried under vacuum. Column chromatographic purification of the crude product [Neutral deactivated alumina, eluent: chloroform/methanol (10:1)] afforded N¹,N²-Ethylenediaminebis [cholic acid amide] (2) (0.81 g, 96%). It was further recrystallized from methanol/chloroform. Its characteristics are as follows: Mp=205° C. (colourless powder). Its spectral analysis is as follows: IR(Nujol): 3344 bs, 2921 bs, 1656 d cm⁻¹. ¹H NMR (500 MHz, CD₃OD+CDCl₃): 4.01 (bs, 2×H—C (12, 12′)), 3.84 (s, 2×H—C (7, 7′)), 3.40 (m, 2×H—C (3, 3′)), 3.33 (bs, 4×H—C (25, 25′)), 1.01 (d, J=5 Hz, 2×CH₃—C (20, 20′)), 0.89 (s, 2×CH₃—C (13, 13′)), 0.69 (s, 2×CH₃—C (10, 10′)). ¹³C NMR (125 MHz, CD₃OD+CDCl₃): 176.76, 73.35, 71.97, 68.68, 46.80, 46.66, 42.02, 41.74, 39.71, 39.53, 35.96, 35.59, 35.06, 34.85, 33.24, 33.08, 32.21, 30.19, 28.34, 27.95, 26.73, 23.52, 22.71, 17.48, 12.68. MS (LCMS, methanol, water, and ammonium acetate, m/z): 859.02 ([M+NH₄]⁺, 38), 842.02 ([M+H]⁺, 100), 452.04 (34), 420.04 (38), 149.03 (34). [α]²⁵=+18.36 (c=1.526, methanol).

### EXAMPLE 2

N-succinimidyl ester (5) (0.98 g, 2 mmol) of deoxycholic acid was dissolved in 2 ml of dimethylformamide. To it ethylenediamine (0.073 ml, 1.1 mmol) was added at 10° C. and reaction mixture was stirred for the period of 3 h at 30° C. Reaction was quenched by the addition of crushed ice. Solid crude product was filtered, washed with cold water and dried under vacuum. Column chromatographic purification of the crude product [Neutral deactivated alumina, eluent: chloroform/methanol (20:1)] afforded N¹,N²-Ethylenediaminebis [deoxycholic acid amide] (3) (0.79 g, 97%). It was further recrystallized from methanol/chloroform. Its characteristics are as follows: Mp=170° C. (colourless powder). Its spectral analysis is as follows: IR (Nujol): 3303 bs, 2921 bs, 1653 d cm⁻¹. ¹H NMR (500 MHz, CD₃OD+CDCl₃): 3.97 (bs, 2×H—C (12, 12′)), 3.56 (m, 2×H—C (3, 3′)), 3.31 (m, 4×H—C (25, 25′)), 0.99 (d, J=5 Hz, 2×CH₃—C (20, 20′)), 0.90 (s, 2×CH₃—C (13, 13′)), 0.68 (s, 2×CH₃—C (10, 10′)). ¹³C NMR (125 MHz, CD₃OD+CDCl₃): 175.66, 72.85, 71.29, 47.89, 46.57, 46.29, 41.96, 39.16, 35.93, 35.82, 35.35, 35.16, 33.99, 33.38, 33.00, 31.53, 29.81, 28.33, 28.33, 27.43, 27.00, 26.05, 23.59, 22.89, 16.99, 12.47. MS (LCMS, methanol, water, and ammonium acetate, m/z): 827.02 ([M+NH₄]⁺, 30), 810.02 ([M+H]⁺, 100), 538.03 (11), 420.04 (14), 240.05 (11), 149.03 (70). [α]²⁵=+46.61 (c=0.472, methanol).

### EXAMPLE 3

This example illustrates antiproliferative activity of the compounds of the invention.

Pharmacological Evaluation

Antiproliferative Activity, Materials and methods: Human oral squamous carcinoma HEp-2 and human mammary adenocarcinoma (MCF-7) cell lines were obtained from National Centre for Cell Science, Pune, India. Cells were maintained as a monolayer in nutrient media MEM supplemented with heat inactivated fetal bovine serum (HyClone, Utah, USA) (10%), penicillin (100 U/ml) and streptomycin (100 μg/ml) (Invitrogen Life Technologies, MD, USA). The cells were grown at 37° C. in 5% CO₂ and humidified air atmosphere. Stock solutions of all the compounds were prepared in DMSO at a concentration of 10-11.5 mM and afterwards diluted to the required concentration. The 3-(4,5-dimethylthiazol-2-yl)-2,5-diphenyltetrazolium bromide (MTT) was dissolved (1 mg/ml) in MEM (without phenol red) and filtered through a Millipore filter, 0.22 μm, before use.

MTT Cell Proliferation Assay: (HEp-2) and (MCF-7) cells were plated at a density of 15,000 cells per well in 96 well tissue culture plates. Cells were allowed to adhere for 24 h at 37° C. and then treated with various concentrations (0, 0.1, 1.0, and 10 μM) of compounds dissolved in DMSO for additional 72 h, in triplicates. In the control wells nutrient medium with correspondent concentration of DMSO only was added to the cells. Thereafter cell proliferation was assessed by replacing treatment medium with 50 μL media containing 1 mg/mL MTT and incubated for 4 h at 37° C. Medium was then aspirated off and formazan crystals were solubilized in 50 mL of isopropanol. The optical density was read on a microplate reader at 570 nm using 630 nm as a reference filter against a blank prepared from cell free wells. Absorbance given by cells treated with the carrier DMSO alone was taken as 100% cell growth. All assays were performed in triplicates.

Antiproliferative Activity. The antiproliferative activity of both the compounds 2 and 3 were tested against human cancer cells, HEp-2 and MCF-7 which were grown in vitro. Compound (2) up to a concentration of 10 μM had no effect on the survival of HEp-2 and MCF-7 cells. Compound (3) elicited a significant reduction in proliferation of both the cell lines and at 10 μM concentration totally inhibited their growth. IC₅₀ value for compound (2) was between 2-3 μM.

We claim:

1. Steroidal dimers having structural formula (1) wherein R is OH or R 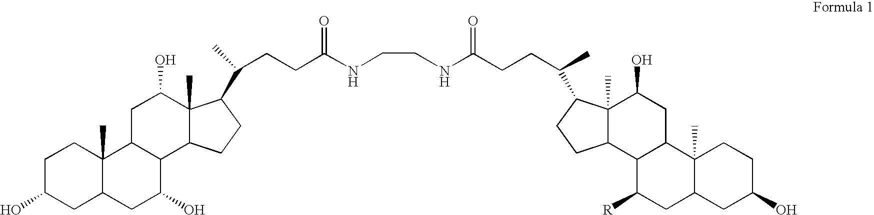
possessing amphiphilic topology as shown below and showing antiproliferative activity.

2. Steroidal dimer as claimed in claim 1 wherein R is OH and of the structural formula (2)

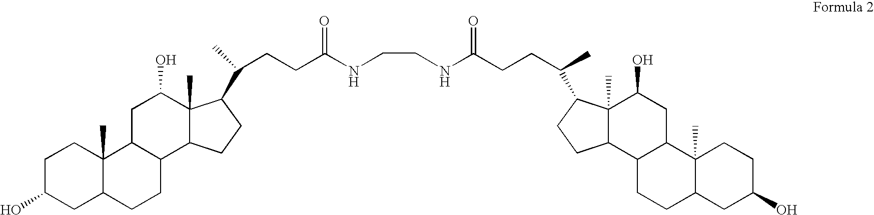

3. Steroidal dimer as claimed in claim 1 wherein R is H and of the structural formula (3)

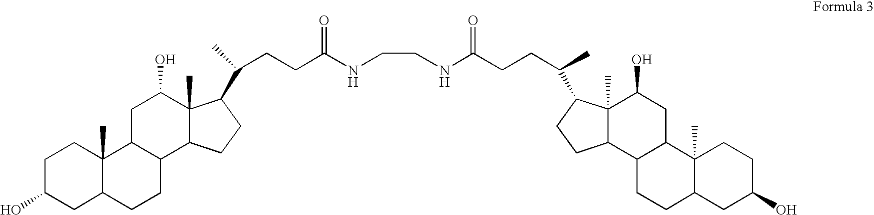

4. A process for the preparation of steroidal dimers having structural formula (1) 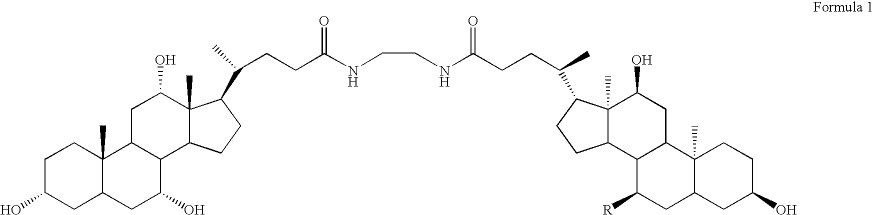
possessing amphiphilic topology as shown below and showing antiproliferative activity which comprises preparing a solution of N-succinimidyl ester of bile acids in an organic solvent, adding to this solution ethylenediamine, stirring the reaction mixture, quenching the reaction mixture with ice, filtering the solid to obtain crude dimers having structural formula (1), further purifying the crude dimers (1) to obtain the pure steroidal dimers (1).

5. A process as claimed in claim 4 wherein R is OH and the steroidal dimer has the structural formula (2)

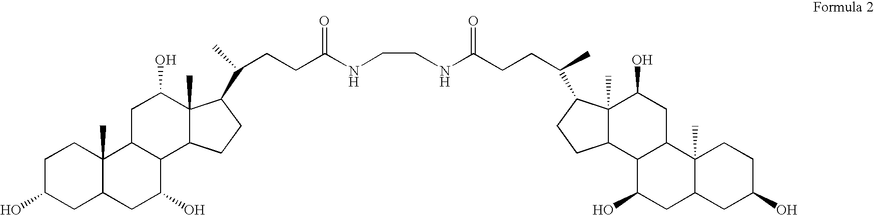

6. A process as claimed in claim 4 wherein R is HH and the steroidal dimer has the structural formula (3)

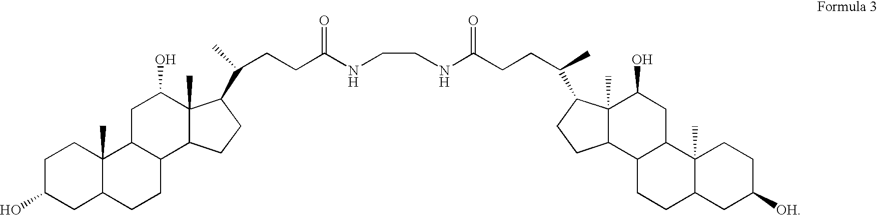

7. A process as claimed in claim 1 wherein the bile acid is selected from the group consisting of cholic acid and deoxycholic acid.

8. A process as claimed in claim 1 wherein the organic solvent used for the preparation of N-succinimidyl ester solution is a chlorinated solvent selected from the group consisting of chloroform and dichloromethane or a polar aprotic solvent selected in turn from the group consisting of dimethy formamide or dimethyl sulfoxide.

9. A process as claimed in claim 1 wherein the crude dimer (1) is purified by column chromatography using silica gel, basic alumina or neutral alumina as adsorbants and ethylacetate-hexane, ethylacetate, ethylacetate-chloroform, chloroform-methanol as solvent systems.

10. A process as claimed in claim 1 wherein the solution of N-succinimidyl ester of bile acids in an organic solvent is prepared at a temperature ranging between 10 to 50° C.

11. A process as claimed in claim 1 wherein the ethylenediamine is added to the solution of N-succinimidyl ester of bile acids at a temperature ranging between 10 to 50° C.

12. A process as claimed in claim 1 wherein the reaction mixture is stirred for a period of at least 1 h at a temperature ranging from 20 to 70° C.

13. A pharmaceutical composition comprising a pharmaceutically effective amount of dimer of formula 1 wherein R is OH or H and a pharmacetically acceptable additive. 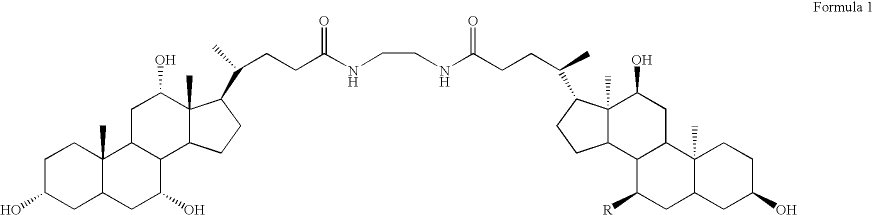
possessing amphiphilic topology as shown below and showing antiproliferative activity.

14. A composition as claimed in claim 13 wherein in the steroidal dimer R is OH and the steroidal dimer has the structural formula (2)

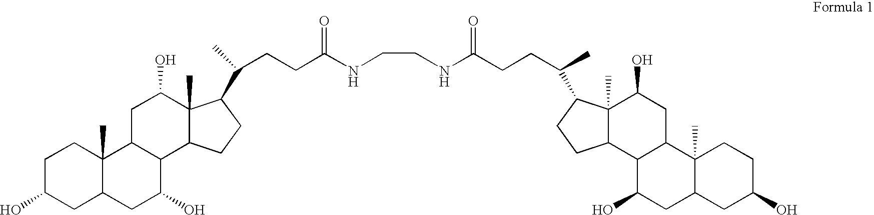

15. A composition as claimed in claim 13 wherein in the steroidal dimer R is H and the steroidal dimer has the structural formula (3)

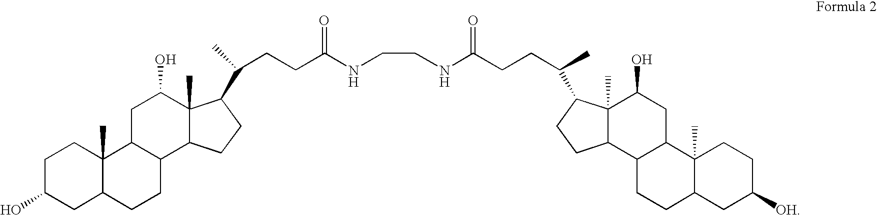

16. Method for the treatment of cancer lines selected from the group consisting of human cancer cells Hep-2 and MCF-7 comprising administering to a patient suffering from the same, an effective amount of steroidal dimer of structural formula 1

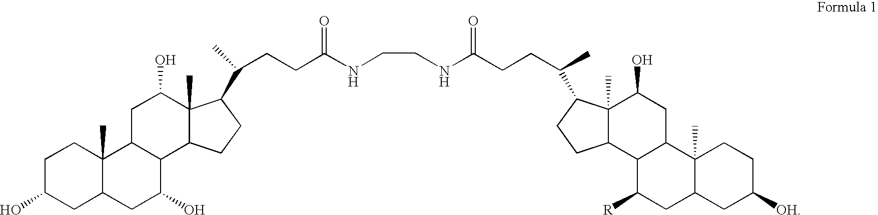

17. Method as claimed in claim 16 wherein the amount of compound of formula (1) is in the range of 2 to 10 μM.
